(19) United States
(12) Patent Application Publication
(10) Pub. No.: US 2022/0241594 A1
(43) Pub. Date: Aug. 4, 2022

(54) Systems and Methods for Deep Brain Stimulation Using Beta Burst Feedback

(71) Applicants: The Board of Trustees of the Leland Stanford Junior University, Stanford, CA (US); University of Washington, Seattle, WA (US)

(72) Inventors: Helen Bronte-Stewart, Stanford, CA (US); Matthew Petrucci, Stanford, CA (US); Ross Anderson, Stanford, CA (US); Raumin Neuville, Stanford, CA (US); Jordan Parker, Stanford, CA (US); Jeffrey Herron, Seattle, WA (US)

(73) Assignees: The Board of Trustees of the Leland Stanford Junior University, Stanford, CA (US); University of Washington, Seattle, WA (US)

(21) Appl. No.: 17/649,668

(22) Filed: Feb. 1, 2022

Related U.S. Application Data
(60) Provisional application No. 63/144,427, filed on Feb. 1, 2021.

(51) Int. Cl. A61N 1/36; A61N 1/05

(52) U.S. Cl. CPC A61N 1/36139; A61N 1/0534; A61N 1/36067; A61N 1/36167; A61N 1/36157

(57) ABSTRACT

Systems and methods for deep brain stimulation using beta burst feedback in accordance with embodiments of the invention are illustrated. One embodiment includes a deep brain stimulation system, including a neurostimulator, and a controller, where the controller is communicatively coupled to the neurostimulator and configured to obtain a plurality of neural activity signals from the neurostimulator, identify beta bursts within each neural activity signal, classify identified beta bursts as pathological or normal, and modify stimulation provided by the neurostimulator based on the classified beta bursts.

### CROSS REFERENCE TO RELATED APPLICATIONS

The present invention claims priority to U.S. Provisional Patent Application Ser. No. 63/144,427 entitled “Systems and Methods for Closed-Loop Deep Brain Stimulation” filed Feb. 1, 2021, the disclosure of which is herein incorporated by reference in its entirety for all purposes.

### FIELD OF THE INVENTION

The present invention generally relates to using beta bursts to modify deep brain stimulation parameters to maintain therapeutic benefit.

### BACKGROUND

Deep brain stimulation (DBS) is a neurosurgical treatment involving the placement of a neurostimulator (pulse generator) which sends electrical impulses through implanted electrodes to a specific target in the brain in order to treat a neurological condition. A common use of DBS is in the treatment of movement disorders. Movement disorders are a class of syndromes which have symptoms of involuntary excess or paucity of movement. Parkinson's disorder (PD) is a condition which often eventually requires DBS for management of symptoms.

PD is a long-term degenerative disorder of the central nervous system which strongly affects the motor system. The four cardinal signs of PD are tremor, slowness of movement (bradykinesia), rigidity, and postural instability. Freezing of gait (FoG) is another symptom of PD in which the patient is unable to move their feet forward despite intending to walk. FoG can cause serious falls as the freezing episode can occur suddenly while the person still has forward momentum. FoG is sometimes non-responsive to medication which can otherwise alleviate other PD symptoms.

### SUMMARY OF THE INVENTION

Systems and methods for deep brain stimulation using beta burst feedback in accordance with embodiments of the invention are illustrated. One embodiment includes a deep brain stimulation system, including a neurostimulator, and a controller, where the controller is communicatively coupled to the neurostimulator and configured to obtain a plurality of neural activity signals from the neurostimulator, identify beta bursts within each neural activity signal, classify identified beta bursts as pathological or normal, and modify stimulation provided by the neurostimulator based on the classified beta bursts.

In another embodiment, the neural activity signals are local field potential signals.

In a further embodiment, to identify beta bursts, the controller is further configured to filter the neural activity signals to remove components outside of the beta band of 13-30 Hz, and identify portions of the filtered signal that exceed a threshold power level.

In still another embodiment, the filter is a finite impulse response filter.

In a still further embodiment, beta bursts exceeding 300 ms are classified by the controller as pathological beta bursts.

In yet another embodiment, to identify beta bursts, the controller is further configured to filter the neural activity signals using a 6 Hz band centered on peak beta power, square the filtered signal, locate peaks in the filtered signal, perform a linear interpolation between the peaks to create a linear envelope, and identify beta bursts based on the average trough power in the linear envelope in the 45-65 Hz band.

In a yet further embodiment, the stimulation is modified using a stimulation map.

In another additional embodiment, the stimulation map comprises the following changes in stimulation parameters: decrease stimulation intensity when a normal beta burst is identified, maintain stimulation intensity when a previously identified beta burst has terminated but the time since cessation of the previously identified beta burst has not exceeded a termination delay threshold, increase stimulation intensity when a pathological beta burst is identified.

In a further additional embodiment, the termination delay threshold is 10 ms.

In another embodiment again, the stimulation intensity is bounded between 2 mA and 5 mA.

In a further embodiment again, a method of deep brain stimulation includes obtaining a plurality of neural activity signals from a neurostimulator using a controller, identifying beta bursts within each neural activity signal using the controller, classifying identified beta bursts as pathological or normal using the controller, and modifying stimulation provided by the neurostimulator based on the classified beta bursts using the controller.

In still yet another embodiment, the neural activity signals are local field potential signals.

In a still yet further embodiment, identifying beta bursts includes filtering the neural activity signals to remove components outside of the beta band of 13-30 Hz, and identifying portions of the filtered signal that exceed a threshold power level.

In still another additional embodiment, the filter is a finite impulse response filter.

In a still further additional embodiment, beta bursts exceeding 300 ms are classified by the controller as pathological beta bursts.

In still another embodiment again, identifying beta bursts further includes filtering the neural activity signals using a 6 Hz band centered on peak beta power, squaring the filtered signal, locating peaks in the filtered signal, performing a linear interpolation between the peaks to create a linear envelope, and identifying beta bursts based on the average trough power in the linear envelope in the 45-65 Hz band.

In a still further embodiment again, the stimulation is modified using a stimulation map.

In yet another additional embodiment, the stimulation map includes the following changes in stimulation parameters: decrease stimulation intensity when a normal beta burst is identified, maintain stimulation intensity when a previously identified beta burst has terminated but the time since cessation of the previously identified beta burst has not exceeded a termination delay threshold, increase stimulation intensity when a pathological beta burst is identified.

In a yet further additional embodiment, the termination delay threshold is 10 ms.

In yet another embodiment again, the stimulation intensity is bounded between 2 mA and 5 mA.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a diagram illustrating a DBS system in accordance with an embodiment of the invention.

FIG. 2 is a block diagram for a DBS controller in accordance with an embodiment of the invention.

FIG. 3 is a flow chart illustrating a method for DBS using beta burst feedback in accordance with an embodiment of the invention.

FIG. 4 is a stimulation map in accordance with an embodiment of the invention.

FIG. 5 is a set of two charts, where chart A shows stimulation intensity based on beta burst feedback, and chart B shows a closer view of a change in stimulation intensity based on beta burst feedback in accordance with an embodiment of the invention.

FIG. 6 shows a complete stack of example input signal, processed input signal, state classifications, and output signal in accordance with an embodiment of the invention.

### DETAILED DESCRIPTION

Movement disorders tend to have severe and chronic impacts on the lives of patients who have them. While some movement symptoms can be treated with drugs, some conditions such as Parkinson's disorder (PD) tend to become drug-resistant after several years. When other interventions become unworkable, deep brain stimulation (DBS) becomes a more viable treatment option.

Many DBS systems are “open-loop” which means that stimulation settings are manually input by a medical professional. DBS stimulation settings conventionally are clinically set by a medical professional during a process known as “programming.” Some DBS systems enable different stimulation profiles to be optionally loaded by the medical professional that can be manually switched between by a patient between programming sessions. Some “closed-loop” DBS systems also exist which acquire biomarkers and can modify stimulation in response in order to have a more flexible system during real-time operation.

DBS systems described herein can use beta bursts occurring within the brain to recognize onset of a motor disability event, gait impairment, and/or freezing of gait (FoG), and modify DBS stimulation parameters in order to restore normal movement. In many embodiments, systems and methods described herein can predict these symptomatic movement events and preemptively modify stimulation parameters in order to preclude the onset of the symptom. Beta bursts are periods of high intensity activity occurring within the beta band of 13-30 Hz within the brain. In many embodiments, a threshold intensity is determined above which beta band oscillations are considered a beta burst.

Based on the presence (or lack thereof) of beta bursts, stimulation parameters can be modified. Simulation parameters can include (but are not limited to) pulse frequency, pulse amplitude (i.e. intensity), and pulse train patterns. In some embodiments, the changes in stimulation parameters are made according to a stimulation map which indicates a change in one or more stimulation parameters in response to a classification of the current beta band activity. DBS systems using beta burst feedback are discussed in further detail below.

### DBS Systems Using Beta Burst Feedback

DBS systems described herein can record neural activity in the brain via one or more electrodes and process the neural activity signals to modify stimulation in real time. In numerous embodiments, the neural activity is recorded as local field potentials (LFPs). In various embodiments, the neural activity signals can be streamed to an external controller which can classify the neural activity signal and trigger a neurostimulator to modify the stimulation it is providing. However, in some embodiments, the implanted neurostimulator can perform the functions of the external controller, resulting in a fully implanted closed-loop system.

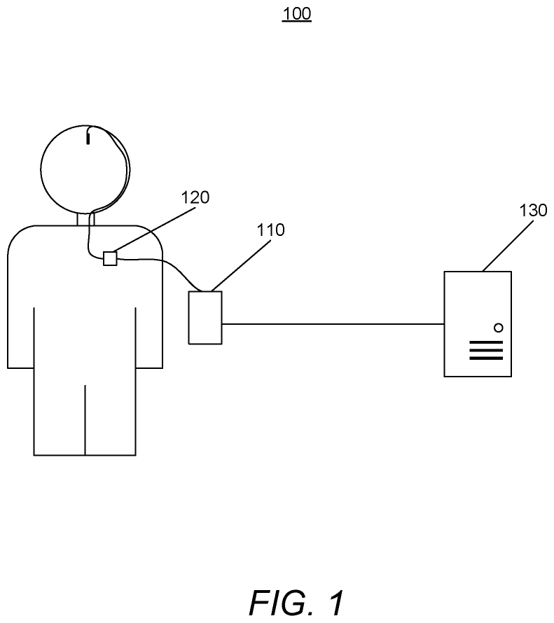

Turning now to FIG. 1, a DBS system using beta burst feedback in accordance with an embodiment of the invention is illustrated. Controller 110 is communicatively coupled to a neurostimulator 120. In numerous embodiments, the controller communicates with the neurostimulator via a wireless connection. The controller can be implemented using an external device or an internal device. In a variety of embodiments, the controller can be a smart phone, or other portable electronic device. The neurostimulator 120 provides electrical stimulation to a patient's brain via electrodes connected to the neurostimulator via one or more leads. The neurostimulator can further record neural activity signals via electrodes implanted into the brain. In some embodiments, the electrodes used for stimulation and recording are the same, although different electrodes can be used as appropriate to the requirements of specific applications of embodiments of the invention. In some embodiments, the controller and neurostimulator are integrated as a single implantable device.

Controller 110 also communicates with a programming device 130. In many embodiments, the programming device 130 is a computing device capable of providing updates to the controller. Updates can include new stimulation maps, new parameter settings, new predictive models, and/or any other type of update to the operation of the controller as needed. In various embodiments, the controller can provide data regarding its operation to the programming device for review by medical professionals. In many embodiments, controllers are capable of directly receiving programming via manual input without a programming device. Controllers and programming devices can be networked together. In many embodiments, the connection is wireless, although wired connections can be used instead or as well. Wireless connections can be made over networks such as (but not limited to) the Internet.

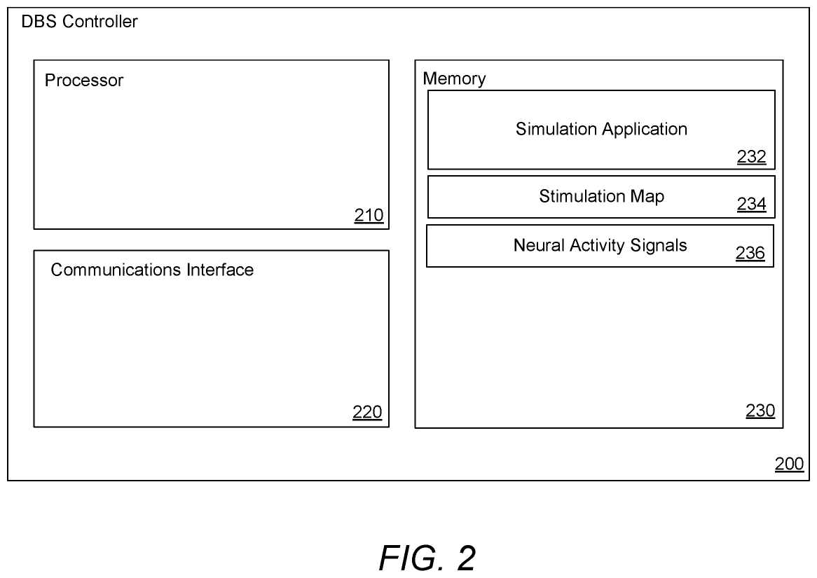

Turning now to FIG. 2, a block diagram for a controller in accordance with an embodiment of the invention is illustrated. DBS controller 200 includes a processor 210. Processor 210 can be any type of computational processing unit, including, but not limited to, microprocessors, central processing units, graphical processing units, parallel processing engines, or any other type of processor as appropriate to the requirements of specific applications of embodiments of the invention. Processors can include multiple processors and/or multiple types of processing architectures.

Controller 200 further includes a communications interface capable of communicating with neurostimulators, programming devices, and/or any other device via appropriate communication methodologies as appropriate to the requirements of specific applications of embodiments of the invention. The controller 200 additionally includes a memory 230. The memory can be made of volatile and/or non-volatile memory. The memory 230 contains a stimulation application which is capable of directing the processor to generate stimulation parameters based on neural activity signals received from the neurostimulator and provide said parameters back to a neurostimulator. The memory 230 can also contain a stimulation map 234 which contains instructions on how to modify stimulation parameters in response to the probability of a detected abnormal movement event. In various embodiments, the memory 230 further contains one or more neural activity signals 236 obtained from the neurostimulator.

While a specific system architecture and controller architecture are illustrated in FIGS. 1 and 2 respectively, as can readily be appreciated, any number of different architectures can be used as appropriate to the requirements of specific applications of embodiments of the invention. For example, controllers and neurostimulators can be integrated into the same hardware. By way of further example, different electrodes (or entirely different neural activity signal recording devices can be used as appropriate to the requirements of specific applications of embodiments of the invention. Processes for modifying stimulation parameters based on kinematic data are discussed in further detail below.

### DBS Using Beta Burst Feedback

Processes for modifying DBS parameters using beta burst feedback enable real-time closed-loop response to the changing condition of a patient. In many embodiments, abnormal movement events are identified by processing neural activity signals recorded in the patient's brain. The neural activity signal can be filtered to isolate the beta band. In various embodiments, a bandpass finite impulse response (FIR) filter is used with patient-specific coefficients to isolate the beta band. While a FIR has advantages in that it can improve robustness against possible dropped packets, other bandpass filters can be used without departing from the scope or spirit of the invention.

In various embodiments, the beta band signal is filtered using a 6 Hz band centered on the peak beta power measured using a spectral density analysis. A linear envelope can be calculated by squaring the data, finding the peaks, and linearly interpolating between the peaks. Beta bursts can then be defined based on the average trough power in the linear envelope of the signal in the 45-65 Hz band.

The beta band signal can be analyzed, and where the power of the signal is above a patient identified threshold, a beta burst can be identified. Beta bursts can further be classified as either “pathological” or “normal”. In many embodiments, a pathological burst is defined as being longer than 300 ms. A stimulation map which relates signal classifications to changes in stimulation parameters can be used to tune the DBS.

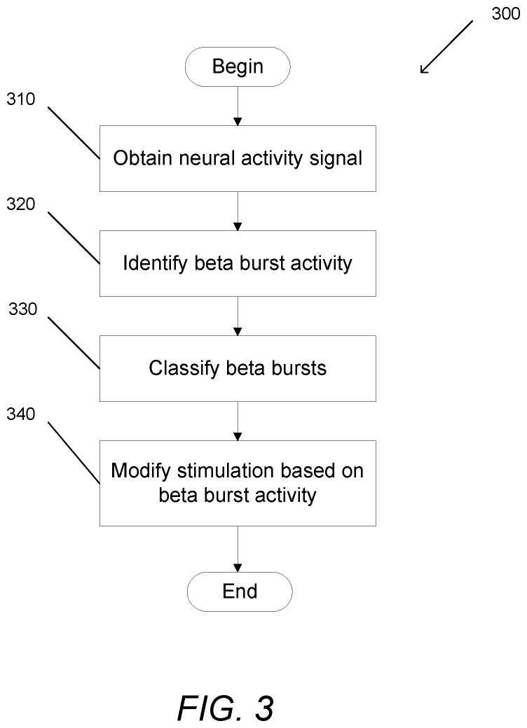

Turning now to FIG. 3, a process for modifying stimulation parameters in accordance with an embodiment of the invention is illustrated. Process 300 includes obtaining (310) a neural activity signal using the neurostimulator. In many embodiments, the neural activity signal is LFP over time. Beta burst activity is identified (320) in the neural activity signal and the identified beta bursts are classified (330) as normal or pathological. Depending on whether or not there is currently no beta burst, a normal beta burst, or a pathological beta burst, stimulation provided by the neurostimulator is modified (340). In many embodiments, a stimulation map is used to guide the modification. However, other data structures and/or methods can be used to trigger the responses as appropriate to the requirements of specific applications of embodiments of the invention.

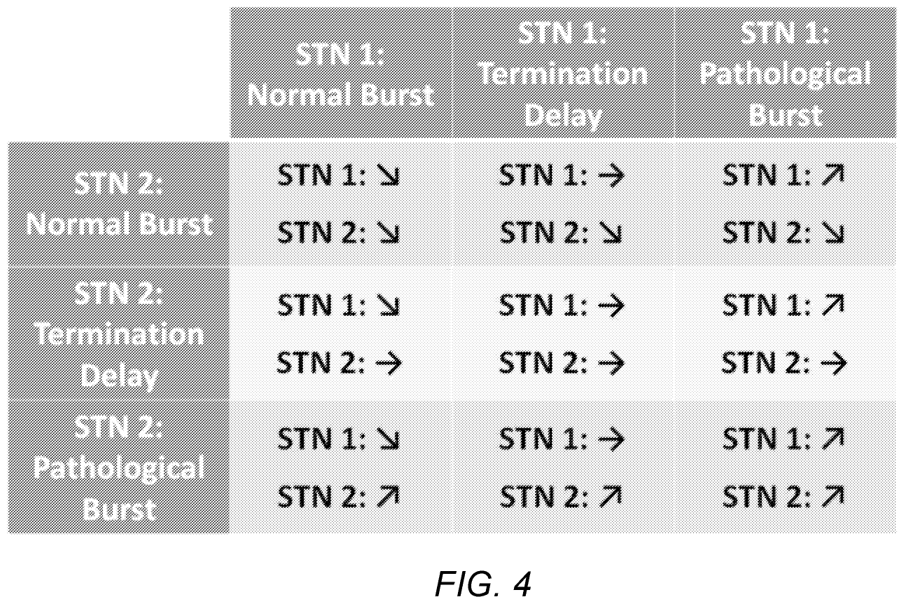

Turning now to FIG. 4, a stimulation map in accordance with an embodiment of the invention is illustrated. In the illustrated stimulation map, the patient has two different electrodes implanted, one in each (left and right) subthalamic nucleus (STN), labeled STN 1 and STN 2. In this case, two different neural activity signals are obtained, one from each STN. Therefore, each STN signal independently is processed to classify beta bursts for that location. The matrix illustrated shows how stimulation for each STN should be changed based on the current state of each STN.

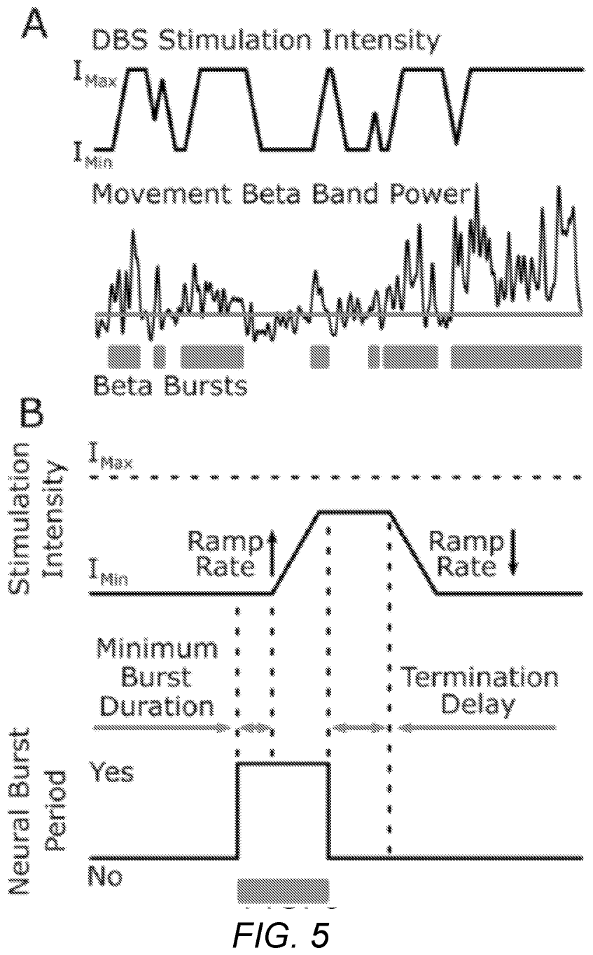

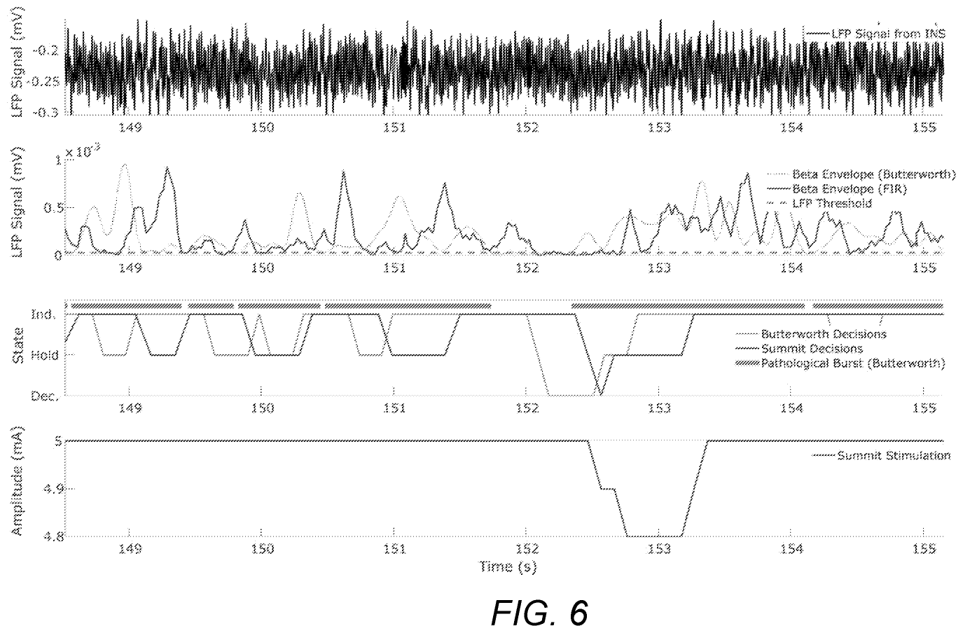

Turning now to FIG. 5, two charts are illustrated where chart A shows stimulation intensity compared to an isolated beta band signal, and chart B graphically depicts the timing and rationale for the stimulation changes at a particular small window of the signal in accordance with an embodiment of the invention. Turning now to FIG. 6, a full stack (top to bottom) of example input LFP signal, beta band signal, beta burst classification, and resultant stimulations in accordance with an embodiment of the invention are illustrated. While a particular process is illustrated in FIG. 3, and specific examples are shown in FIGS. 4, 5, and 6, as can be readily appreciated, multiple variations can be made without departing from the scope or spirit of the invention. For example, different thresholds for different patients can be used, different filtering techniques can be used, different signal recording techniques can be used, any/or any other modification which maintains use of beta bursts for closed loop control of DBS can be used as appropriate to the requirements of specific applications of embodiments of the invention.

Although specific systems and methods for DBS using beta burst feedback are discussed above, many different systems and methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A deep brain stimulation system, comprising:
a neurostimulator; and
a controller, where the controller is communicatively coupled to the neurostimulator and configured to:
obtain a plurality of neural activity signals from the neurostimulator;
identify beta bursts within each neural activity signal;
classify identified beta bursts as pathological or normal; and
modify stimulation provided by the neurostimulator based on the classified beta bursts.

2. The deep brain stimulation system of claim 1, wherein the neural activity signals are local field potential signals.

3. The deep brain stimulation system of claim 1, wherein to identify beta bursts, the controller is further configured to:
filter the neural activity signals to remove components outside of the beta band of 13-30 Hz; and
identify portions of the filtered signal that exceed a threshold power level.

4. The deep brain stimulation system of claim 3, wherein the filter is a finite impulse response filter.

5. The deep brain stimulation system of claim 3, wherein beta bursts exceeding 300 ms are classified by the controller as pathological beta bursts.

6. The deep brain stimulation system of claim 3, wherein to identify beta bursts, the controller is further configured to:
filter the neural activity signals using a 6 Hz band centered on peak beta power;
square the filtered signal;
locate peaks in the filtered signal;
perform a linear interpolation between the peaks to create a linear envelope; and
identify beta bursts based on the average trough power in the linear envelope in the 45-65 Hz band.

7. The deep brain stimulation system of claim 1, wherein the stimulation is modified using a stimulation map.

8. The deep brain stimulation system of claim 1, wherein the stimulation map comprises the following changes in stimulation parameters:
decrease stimulation intensity when a normal beta burst is identified;
maintain stimulation intensity when a previously identified beta burst has terminated but the time since cessation of the previously identified beta burst has not exceeded a termination delay threshold; and
increase stimulation intensity when a pathological beta burst is identified.

9. The deep brain stimulation system of claim 8, wherein the termination delay threshold is 10 ms.

10. The deep brain stimulation system of claim 8, wherein the stimulation intensity is bounded between 2 mA and 5 mA.

11. A method of deep brain stimulation, comprising:
obtaining a plurality of neural activity signals from a neurostimulator using a controller;
identifying beta bursts within each neural activity signal using the controller;
classifying identified beta bursts as pathological or normal using the controller; and
modifying stimulation provided by the neurostimulator based on the classified beta bursts using the controller.

12. The method of deep brain stimulation of claim 11, wherein the neural activity signals are local field potential signals.

13. The method of deep brain stimulation of claim 11, wherein identifying beta bursts comprises:
filtering the neural activity signals to remove components outside of the beta band of 13-30 Hz; and
identifying portions of the filtered signal that exceed a threshold power level.

14. The method of deep brain stimulation of claim 13, wherein the filter is a finite impulse response filter.

15. The method of deep brain stimulation of claim 13, wherein beta bursts exceeding 300 ms are classified by the controller as pathological beta bursts.

16. The method of deep brain stimulation of claim 13, wherein identifying beta bursts further comprises:
filtering the neural activity signals using a 6 Hz band centered on peak beta power;
squaring the filtered signal;
locating peaks in the filtered signal;
performing a linear interpolation between the peaks to create a linear envelope; and
identifying beta bursts based on the average trough power in the linear envelope in the 45-65 Hz band.

17. The method of deep brain stimulation of claim 11, wherein the stimulation is modified using a stimulation map.

18. The method of deep brain stimulation of claim 11, wherein the stimulation map comprises the following changes in stimulation parameters:
decrease stimulation intensity when a normal beta burst is identified;
maintain stimulation intensity when a previously identified beta burst has terminated but the time since cessation of the previously identified beta burst has not exceeded a termination delay threshold; and
increase stimulation intensity when a pathological beta burst is identified.

19. The method of deep brain stimulation of claim 18, wherein the termination delay threshold is 10 ms.

20. The method of deep brain stimulation of claim 18, wherein the stimulation intensity is bounded between 2 mA and 5 mA.
